(19) United States
(12) Patent Application Publication
(10) Pub. No.: US 2024/0117053 A1
(43) Pub. Date: Apr. 11, 2024

(54) CLEC12A ANTIBODY FRAGMENT SEQUENCES AND METHODS

(71) Applicant: REGENTS OF THE UNIVERSITY OF MINNESOTA, Minneapolis, MN (US)

(72) Inventors: Martin FELICES, Circle Pines, MN (US); Jeffrey S. MILLER, North Oaks, MN (US)

(21) Appl. No.: 17/768,793

(22) PCT Filed: Oct. 14, 2020
(86) PCT No.: PCT/US2020/055468

§ 371 Date: Apr. 13, 2022

Related U.S. Application Data
(60) Provisional application No. 62/916,340, filed on Oct. 17, 2019.

(51) Int. Cl. C07K 16/28; A61K 47/68

(52) U.S. Cl. CPC C07K 16/2851; A61K 47/6849; A61K 39/00

(57) ABSTRACT

An anti-CLEC12A polypeptide generally includes an amino acid sequence having at least 90% amino acid similarity to SEQ ID NO:14. In some embodiments, anti-CLEC12A polypeptide may be incorporated into an anti-CLEC12A biologic. In some of these embodiments, the anti-CLEC12A biologic can be a bi-specific Killer engager molecule (BiKE), a tri-specific Killer engager molecule (TriKE), a tetra-specific Killer engager molecule (TetraKE), a penta-specific Killer engager molecule (PentaKE), a bi-specific T cell engager molecule (BiTE), a tri-specific T cell engager molecule (TriTE), tetra-specific T cell engager molecule (TetraTE), a penta-specific T cell engager molecule (PentaTE), a chimeric antigen receptor, a full antibody, an antibody-drug conjugate (ADC) molecule, a targeted delivery construct, or a labeling construct.

### CROSS-REFERENCE TO RELATED APPLICATION

This application claims the benefit of U.S. Provisional Patent Application No. 62/916,340, filed Oct. 17, 2019, which is incorporated herein by reference in its entirety.

### SEQUENCE LISTING

This application contains a Sequence Listing electronically submitted to the United States Patent and Trademark Office via EFS-Web as an ASCII text file entitled “0110-000633WO01_ST25.txt” having a size of 39 KB and created on Oct. 14, 2020. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### SUMMARY

This disclosure describes, in one aspect, an anti-CLEC12A polypeptide. The anti-CLEC12A polypeptide has an amino acid sequence having at least 90% amino acid similarity to SEQ ID NO:14.

In some embodiments, the anti-CLEC12A polypeptide includes the amino acid sequence of SEQ ID NO:1, the amino acid sequence of SEQ ID NO:2, the amino acid sequence of SEQ ID NO:3, the amino acid sequence of SEQ ID NO:4, the amino acid sequence of SEQ ID NO:5, the amino acid sequence of SEQ ID NO:6, the amino acid sequence of SEQ ID NO:7, the amino acid sequence of SEQ ID NO:8, the amino acid sequence of SEQ ID NO:9, the amino acid sequence of SEQ ID NO:10, the amino acid sequence of SEQ ID NO:11, the amino acid sequence of SEQ ID NO:12, or the amino acid sequence of SEQ ID NO:13.

In some embodiments, the anti-CLEC12A polypeptide includes the amino acid sequence of SEQ ID NO:29, the amino acid sequence of SEQ ID NO:30, and the amino acid sequence of SEQ ID NO:31.

In some embodiments, anti-CLEC12A polypeptide may be incorporated into an anti-CLEC12A biologic. In some of these embodiments, the anti-CLEC12A biologic can be a bi-specific Killer engager molecule (BiKE), a tri-specific Killer engager molecule (TriKE), a tetra-specific Killer engager molecule (TetraKE), a penta-specific Killer engager molecule (PentaKE), a bi-specific T cell engager molecule (BiTE), a tri-specific T cell engager molecule (TriTE), tetra-specific T cell engager molecule (TetraTE), a penta-specific T cell engager molecule (PentaTE), a chimeric antigen receptor, a full antibody, an antibody-drug conjugate (ADC) molecule, a targeted delivery construct, or a labeling construct.

In some embodiments, the anti-CLEC12A biologic may be combined with a pharmaceutically acceptable carrier to form a pharmaceutical composition.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Amino acid sequence alignment of 13 unique anti-CLEC12A antibody variant clones (SEQ ID NOS:1-13), identified by phage display, and the anti-CLEC12A antibody consensus sequence (SEQ ID NO:14). CDR1, CDR2, and CDR3 sequences are underlined.

FIG. 2. SDS-PAGE of His-tagged human CLEC12A extracellular domain used for screening.

FIG. 3. Functional screening of bi-specific compounds containing humanized camelid (huCAM) anti-CLEC12A antibody fragments using PBMCs. Peripheral blood mononuclear cells (PBMCs) were incubated with the CLEC12A-expressing promyelocytic leukemia cell line HL60 in the presence of noted bi-specific compounds containing the different clones (SEQ ID NOs:1-13). A tri-specific compound (SEQ ID NO:15) containing an anti-CLEC12A scFv and known to target CLEC12A was used a positive control, while no treatment (NT) was used as a negative control. After five hours in culture, cells were harvested, stained for surface antigens, fixed, permeabilized, and stained for intracellular interferon gamma (IFNγ). Cells were run on a flow cytometer and NK cell activation was measured via evaluation of degranulation (CD107a) of CD56⁺CD3⁻ NK cells.

FIG. 4. Functional screening of bi-specific compounds containing humanized camelid (huCAM) anti-CLEC12A antibody fragments using PBMCs. Peripheral blood mononuclear cells (PBMCs) were incubated with the CLEC12A-expressing promyelocytic leukemia cell line HL60 in the presence of noted bi-specific compounds containing the different clones (SEQ ID NOs:1-13). A tri-specific compound (SEQ ID NO:15) containing an anti-CLEC12A scFv and known to target CLEC12A was used a positive control, while no treatment (NT) was used as a negative control. After five hours in culture, cells were harvested, stained for surface antigens, fixed, permeabilized, and stained for intracellular interferon gamma (IFNγ). Cells were run on a flow cytometer and NK cell activation was measured via evaluation of IFNγ production by CD56⁺CD3⁻ NK cells.

FIG. 5. Evaluation of a bi-specific compound containing humanized camelid (huCAM) anti-CLEC12A Clone 33. To determine background activation mediated by the Clone 33 bi-specific compound, enriched NK cells were incubated with the bi-specific compound containing anti-CLEC12A Clone 33 (Clone 33 huCAM engager), the CLEC12A tri-specific compound (SEQ ID NO:15; scFv engager), or no compound (NT) for five hours. NK cell degranulation (CD107a, left) and cytokine production (IFNγ, right) were measured by flow cytometry. While the scFv shows some background activation in terms of degranulation, Clone 33 doesn't, highlighting better specificity.

FIG. 6. Evaluation of a bi-specific compound containing humanized camelid (huCAM) anti-CLEC12A Clone 33. To determine activation mediated by the bi-specific compound containing the anti-CLEC12A Clone 33 against a CLEC12-expressing target, enriched NK cells were incubated with HL60 cells and either the bi-specific compound (SEQ ID NO:34) containing anti-CLEC12A Clone 33 (Clone 33 huCAM engager), the CLEC12A tri-specific compound (SEQ ID NO:15; scFv engager), or no compound (NT) for five hours. NK cell degranulation (CD107a, left) and cytokine production (IFNγ, right) were measured by flow cytometry. The Clone 33-containing tri-specific compound induces NK cell degranulation against HL60 targets and induces the same amount of cytokine production as the scFv-containing positive control.

FIG. 7. Evaluation of a bi-specific compound containing humanized camelid (huCAM) anti-CLEC12A Clone 33. To determine activation mediated by the bi-specific compound containing the anti-CLEC12A Clone 33 against a CLEC12-negative target (non-specific activation), enriched NK cells were incubated with Raji (Burkitt's lymphoma) cells and either the bi-specific compound (SEQ ID NO:34) containing anti-CLEC12A Clone 33 (Clone 33 huCAM engager), the CLEC12A tri-specific compound (SEQ ID NO:15; scFv engager), or no compound (NT) for five hours. NK cell degranulation (CD107a, left) and cytokine production (IFNγ, right) were measured by flow cytometry. The Clone 33-containing bi-specific compound induces less non-specific degranulation against CLEC12A-negative targets than the tri-specific compound containing the anti-CLEC12A scFv.

FIG. 8. Evaluation of binding specificity of Clone 33. (A) Binding of Clone 33-containing bi-specific compound (SEQ ID NO:34) and tri-specific compound (SEQ ID NO:15) containing anti-CLEC12A scFv to CLEC12A⁺HL60 cells. (B) Binding of Clone 33-containing bi-specific compound (SEQ ID NO:34) and tri-specific compound (SEQ ID NO:15) containing anti-CLEC12A scFv to CLEC12A-negative Raji cells.

### DETAILED DESCRIPTION OF ILLUSTRATIVE EMBODIMENTS

C-type lectin domain family 12 member A (CLEC12A) is a protein that in humans is encoded by the CLEC12A gene. CLEC12A a member of the C-type lectin/C-type lectin-like domain (CTL/CTLD) superfamily. CLEC12A is an inhibitory C-type lectin-like receptor. It contains an immunoreceptor tyrosine-based inhibitory motif (HIM) in its cytoplasmic tail that can associate with signaling phosphatases such as, for example, SHP-1 and SHP-2.

Human CLEC12A is a monomer expressed primarily on myeloid cells such as, for example, granulocytes, monocytes, macrophages, and dendritic cells. CLEC12A is a target for immunotherapy for treating myeloid malignancies such as, for example, acute myeloid leukemia. (AML) or a myelodysplastic syndrome (MDS) since CLEC12A is expressed on the majority of myeloid blasts and leukemic stem cells (LSCS) myeloid cells but is not expressed on cells of normal tissue or normal hematopoietic stem cells.

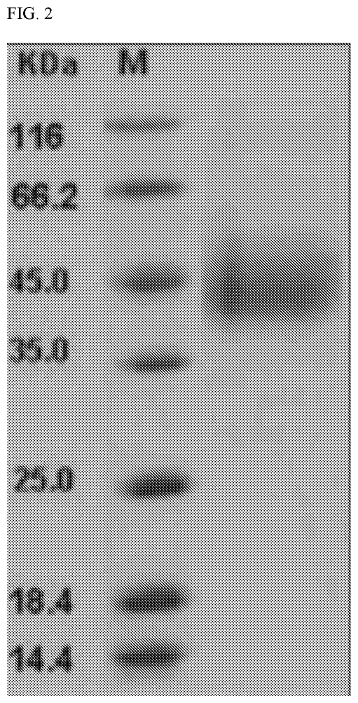

Human CLEC12 was subjected to phage display single domain antibody (sdAb) library screening. Three rounds of library panning were performed using a premade single domain antibody library for human CLEC12A. As shown in FIG. 2, the SDS-PAGE result indicated that the recombinant human CLEC12A protein was of high quality. Biopanning was then performed to enrich the specific binders to the target human CLEC12A. As shown in Table 1, after three rounds of library screening, strong enriching effect was observed for human CLEC12A, and clear difference was found between the target screening groups and no coating control groups.

TABLE 1

| Round | Input | Output | Enriching factor |
|---|---|---|---|
| 1 | 2.00 × 10¹¹ | 5.84 × 10⁴ | 3.42 × 10⁶ |
| 2 - P | 2.44 × 10¹¹ | 2.13 × 10⁵ | 1.15 × 10⁶ |
| 2 - N | 3.05 × 10¹⁰ | 2.02 × 10³ | 1.51 × 10⁷ |
| 3 - P | 2.54 × 10¹¹ | 4.51 × 10⁷ | 5.63 × 10³ |
| 3 - N | 3.18 × 10¹⁰ | 2.40 × 10³ | 1.33 × 10⁷ |

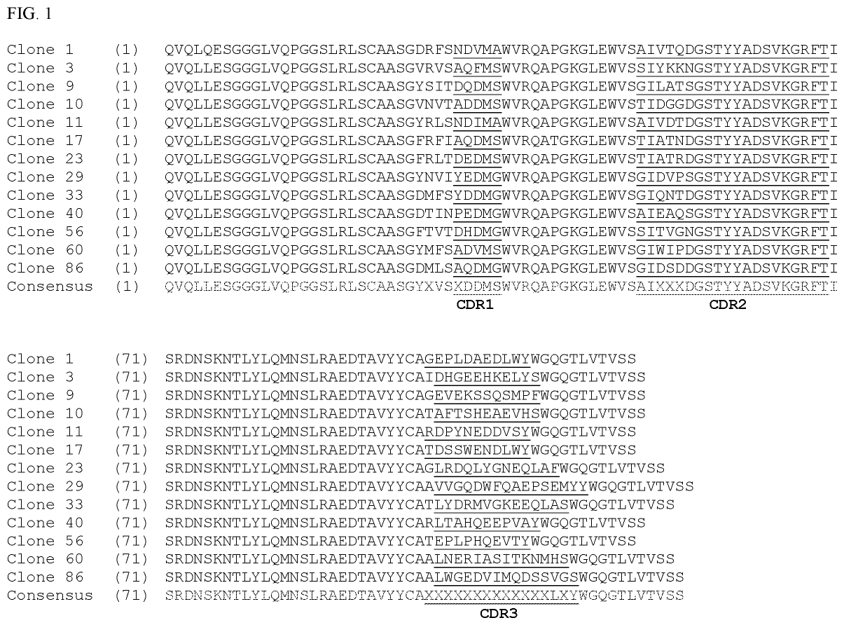

Ninety-six clones were selected from the 3rd-P eluate and subjected to monoclonal phage ELISA. Eighty-two positive clones were identified and subjected to DNA sequencing. Seventy-seven clones were sequenced successfully, and 13 unique sequences were found (SEQ ID NOS:1-13). The 13 unique sequences were aligned, yielding a consensus sequence (SEQ ID NO:14) and identifying complementarity-determining regions (CDRs), as shown in FIG. 1.

After sequencing, the 13 unique sequences were cloned into the soluble VHH-AP expression vector and subjected to soluble expression and soluble ELISA. As shown in Table 2, all 13 clones bound to human CLEC12A positively.

TABLE 2
Monoclonal phage ELISA

| Clone | Induce Temp | CLEC12A | No Coating |
|---|---|---|---|
| 1 | 30° C. | 0.1900 | 0.1740 |
| | 37° C. | 2.0060 | 0.3150 |
| 3 | 30° C. | 0.2240 | 0.1570 |
| | 37° C. | 0.3640 | 0.1870 |
| 9 | 30° C. | 0.3910 | 0.1700 |
| | 37° C. | 0.4720 | 0.2080 |
| 10 | 30° C. | 0.3480 | 0.1830 |
| | 37° C. | 0.6020 | 0.3280 |
| 11 | 30° C. | 1.4340 | 0.3250 |
| | 37° C. | 1.4510 | 0.3650 |
| 17 | 30° C. | 0.9840 | 0.2300 |
| | 37° C. | 0.6100 | 0.2370 |
| 23 | 30° C. | 0.6690 | 0.1590 |
| | 37° C. | 0.7120 | 0.1640 |
| 29 | 30° C. | 1.2190 | 0.2290 |
| | 37° C. | 1.3380 | 0.1750 |
| 33 | 30° C. | 1.5930 | 0.1820 |
| | 37° C. | 1.5190 | 0.1990 |
| 40 | 30° C. | 09.190 | 0.2110 |
| | 37° C. | 0.9040 | 0.3040 |
| 56 | 30° C. | 0.2920 | 0.1860 |
| | 37° C. | 0.4370 | 0.2760 |
| 60 | 30° C. | 0.7130 | 0.2140 |
| | 37° C. | 0.6900 | 0.2920 |
| 86 | 30° C. | 1.6750 | 0.1620 |
| | 37° C. | 1.7080 | 0.1610 |
| TG1 Lysate | — | 0.1430 | 0.1670 |
| TBS | — | 0.1560 | 0.1660 |

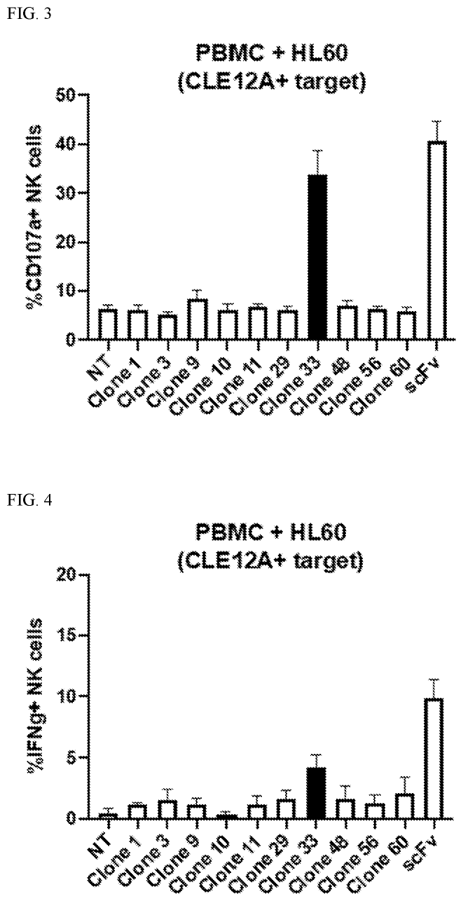

FIG. 3 and FIG. 4 show the functional screening of humanized camelid (huCAM) anti-CLEC12A clones using PBMCs. The humanized anti-CLEC12A camelid clone variants (SEQ ID NOs:1-13) were cloned as the targeting domain into a bi-specific backbone containing a camelid anti-CD16 and a linker. These bi-specific compounds activated natural killer (NK) cells through formation of a cytolytic bridge between the NK cell (through binding of CD16) and the tumor cell through binding of CLEC12A. A tri-specific killer engager (SEQ ID NO:15) previously tested and shown to target CLEC12A through a single chain variable fragment (scFv) was used as a positive control. The data show that Clone 33 displays the highest activity. Some clones were produced in smaller amounts and thus were tested at lower concentrations than the scFv (tri-specific positive control) or the Clone 33 bi-specific compound. Thus, no conclusions can be drawn from results where a clone appears to lack activity compared to the negative control.

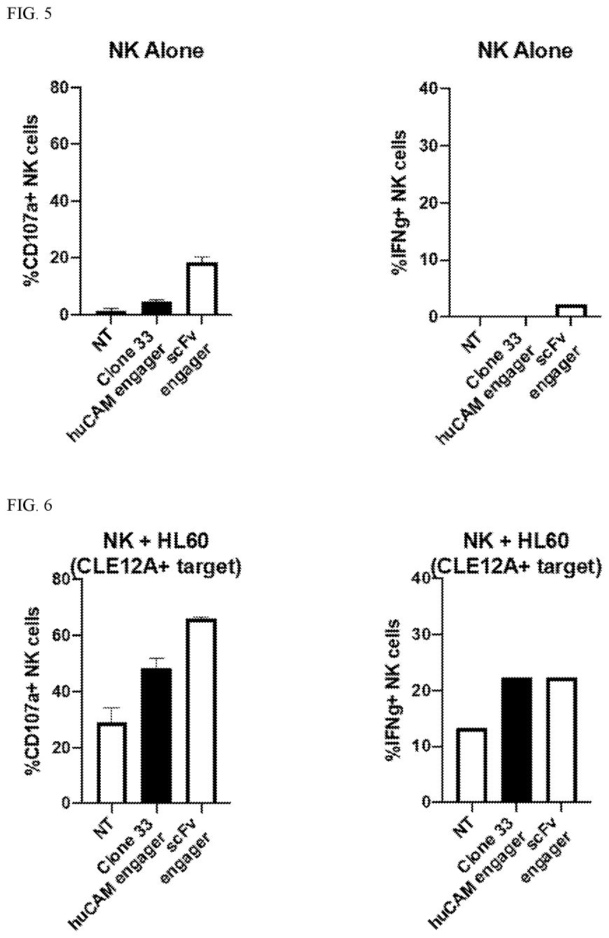

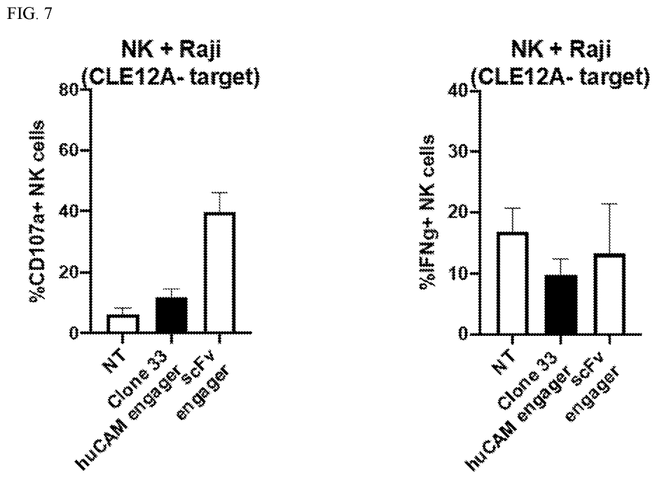

To determine the functional specificity of the humanized CLEC12A camelid Clone 33 antibody fragment, the CD16-Clone 33 bi-specific compound (SEQ ID NO:34) was used. This bi-specific compound has the ability to activate natural killer (NK) cells through formation of a cytolytic bridge between an NK cell (through binding of CD16) and a tumor cell (through binding of CLEC12A). FIG. 5. shows background activation of NK cells as measured by NK cell degranulation (CD107a, left) and induction of IFNγ (right). The bi-specific compound containing Clone 33 shows minimal background NK cell activation. FIG. 6 shows activation of NK cells in the presence of CLEC12A-positive HL60 cells. The bi-specific compound containing the anti-CLEC12A Clone 33 antibody fragment induced both degranulation (left) and production of IFNγ (right), with IFNγ induction being nearly identical to the positive control, the anti-CLEC12A-scFv-containing tri-specific compound (SEQ ID NO:15). FIG. 7. shows NK activation in the presence of CLEC12A-negative Raji (Burkitt's lymphoma) cells. The Clone-33-containing bi-specific compound induces less non-specific degranulation against a CLEC12A-negative target than the anti-CLEC12A-scFv-containing tri-specific compound.

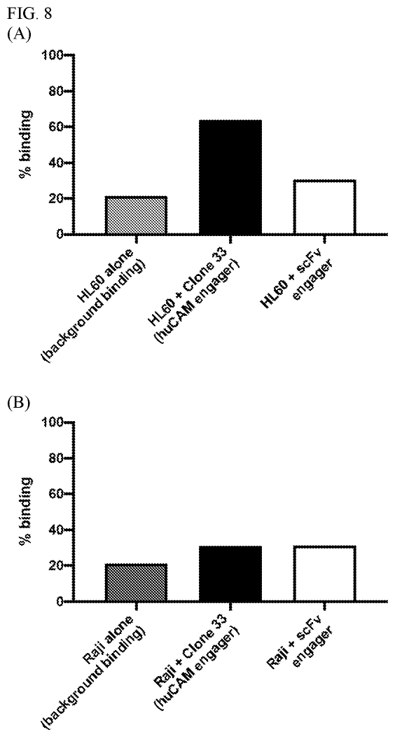

FIG. 8 further shows the binding specificity of the anti-CLEC12A Clone 33. Both the bi-specific compound containing the anti-CLEC12A Clone 33 antibody fragment and the tri-specific compound containing the anti-CLEC12A scFv were constructed in include a 10×HIS tag. Binding of these constructs to CLEC12A-positive HL60 cells (FIG. 8A) and CLEC12A-negative Raji cells (FIG. 8B) was assessed using an anti-HIS-Phycoerythrin-labeled antibody. As a control, basal binding of the anti-HIS-PE antibody was determined without previous binding of the engager compounds (gray bars). As the data shows, both the Clone 33 bi-specific compound and the scFv tri-specific compound induced minimal binding against CLEC12A negative cells. The Clone 33 bi-specific compound induced more binding to the CLEC12A-positive cells than the anti-CLEC12A scFv-containing tri-specific compound.

Thus, this disclosure describes polypeptides that target CLEC12A on myeloid malignancies. Because the polypeptides target CLEC12A, the polypeptides induce less targeting of normal myeloid cells than polypeptides that target other antigens associated with myeloid malignancies such as, for example, CD33. This disclosure expressly describes 12 unique humanized single-domain antibody (sdAb) sequences that target CLEC12A, a consensus sequence derived from an alignment of the 12 unique anti-CLEC12A sdAb sequences, and provides guidance toward additional variants by identifying regions of high conservation and regions of variability among the 13 total sequences.

The anti-CLEC12A polypeptides described herein can be incorporated into biologic constructs that may be used in the context of, for example, therapeutic, diagnostic, and/or detection methods. For example, the anti-CLEC12A polypeptides described herein may be incorporated into a bi-specific Killer engager molecule (BiKE, e.g., SEQ ID NO:34), a tri-specific Killer engager molecule (TriKE, e.g., SEQ ID NO:35), a tetra-specific Killer engager molecule (TetraKE), a penta-specific Killer engager molecule (PentaKE), a bi-specific T cell engager molecule (BiTE), a tri-specific T cell engager molecule (TriTE), a tetra-specific T cell engager molecule (TetraTE), a penta-specific T cell engager molecule (PentaTE), a chimeric antigen receptor (CAR, for expression in, for example, CAR T cells, CAR NK cells, CAR macrophage cells, etc.), a full antibody construct (e.g., to induce antibody-dependent cellular toxicity (ADCC)), an antibody-drug conjugate (ADC) molecule (e.g., for delivery of a toxin), a labeling construct (e.g., for commercial evaluation of antigen expression), a radiolabeled format (e.g., for positron-emission tomography (PET) imaging or directed radiation delivery), an applications for delivery of a cytokine and/or a chemokine, or other general immunotherapeutic approach.

While other reagents—e.g., CD33-binding antibodies and/or antibody fragments—target myeloid malignancies expressing CD33, they also target normal myeloid cells that express lower levels of the target. In contrast, the anti-CLEC12A polypeptides described herein are much less likely to result in on-target, off-tumor toxicity by targeting an antigen that is more specifically expressed on myeloid malignancies (e.g., such as acute myeloid leukemia (AML) cells or myelodysplastic syndromes (MDS) cells). CLEC12A is also expressed in leukemic stem cells. Thus, targeting CLEC12A can limit the likelihood and/or severity of relapse. The anti-CLEC12A polypeptides described herein also may provide advantages over scFvs for forming constructs (e.g., BiKEs, TriKEs, BiTEs, CARs, etc) because sdAbs are more stable than scFvs. Finally, the anti-CLEC12A sequences described herein are all from a humanized library, and thus less likely to rejected in human patients.

This disclosure describes anti-CLEC12A polypeptides. Exemplary anti-CLEC12A polypeptides include polypeptides that include, or are structurally similar to, or are functional variants of, the amino acid sequence of any one of SEQ ID NOs:1-14 or SEQ ID NOs:29-31. SEQ ID NOs:1-13 are variant single domain antibody sequences selected through phage display screening because they specifically bind to CLEC12A. SEQ ID NO:14 is a consensus sequence derived from an alignment analysis of SEQ ID NOs:1-13 (FIG. 1). SEQ ID NO:29 is the consensus sequence of CDR1 as determined from the alignment analysis shown in FIG. 1. SEQ ID NO:30 is the consensus sequence of CDR2 as determined from the alignment analysis shown in FIG. 1. SEQ ID NO:31 is the consensus sequence of CDR3 as determined from the alignment analysis shown in FIG. 1.

As used herein, an anti-CLEC12A polypeptide is “structurally similar” to or a “functional variant” of a reference polypeptide if the amino acid sequence of the anti-CLEC12A polypeptide possesses a specified amount of identity compared to the reference polypeptide. An amino acid sequence is a “functional fragment” of a reference amino acid sequence if the “functional fragment” amino acid sequence contains less than the full-length amino acid sequence of the reference amino acid sequence. A “functional fragment” may further possess a specified amount of sequence identity or sequence specificity compared to the reference amino acid sequence.

Structural similarity and/or sequence identity of two polypeptides can be determined by aligning the amino acid residues of the two polypeptides (for example, a candidate anti-CLEC12A polypeptide and the polypeptide of, for example, any one of SEQ ID NOs:1-14 or SEQ ID NOs:29-31) to optimize the number of identical amino acids along the lengths of their sequences. Gaps in either or both sequences are permitted in making the alignment in order to optimize the number of identical amino acids, although the amino acids in each sequence must nonetheless remain in their proper order. A candidate anti-CLEC12A polypeptide is the polypeptide being compared to the reference polypeptide (e.g., any one of SEQ ID NOs:1-14 or SEQ ID NOs:29-31). A candidate polypeptide can be isolated, for example, from an animal, or can be produced using recombinant techniques, or chemically or enzymatically synthesized.

A pair-wise comparison analysis of amino acid sequences can be carried out using the BESTFIT algorithm in the GCG package (version 10.2, Madison WI). Alternatively, polypeptides may be compared using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in an anti-CLEC12A polypeptide may be selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH₂. Likewise, biologically active analogs of a polypeptide containing deletions or additions of one or more contiguous or noncontiguous amino acids that do not eliminate a functional activity of the polypeptide are also contemplated.

In some embodiments, an anti-CLEC12A polypeptide as described herein can include a polypeptide with at least 90%, at least 91%, at least 92%, at least 93%, at least 94%, at least 95%, at least 96%, at least 97%, at least 98%, or at least 99% amino acid sequence similarity to the reference amino acid sequence.

In some embodiments, an anti-CLEC12A polypeptide as described herein can include at least 90%, at least 91%, at least 92%, at least 93%, at least 94%, at least 95%, at least 96%, at least 97%, at least 98%, or at least 99% amino acid sequence identity to the reference amino acid sequence.

In some embodiments, an anti-CLEC12A polypeptide as described herein also can be designed to provide additional sequences, such as, for example, amino acids added C-terminal or N-terminal of the anti-CLEC12A polypeptide. Such additional amino acids may include, for example, a signal sequence (e.g., SEQ ID NO:32) or a tag that facilitates purification by trapping the tagged anti-CLEC12A polypeptide on a column or by using antibodies. Exemplary tags include, for example, a histidine-rich tag (e.g., SEQ ID NO:33) that allows purification of polypeptides on nickel columns.

This disclosure also describes polynucleotides that encode an anti-CLEC12A polypeptide. Exemplary polynucleotides that encode an anti-CLEC12A polypeptide include the polynucleotides that include the nucleotide sequence of any one of SEQ ID NOs:16-28. SEQ ID NOs:16-28 are, however, merely exemplary. Since the genetic code is well-known, this disclosure describes any polynucleotide that encodes an anti-CLEC12A polypeptide as described herein.

An anti-CLEC12A polypeptide as described herein may be incorporated into a biologic, which is then formulated with a pharmaceutically acceptable carrier. As used herein, an “anti-CLEC12A biologic” is a biologic compound that includes an anti-CLEC12A polypeptide. An anti-CLEC12A biologic may include additional functional moieties depending on the fundamental structural platform of the biologic (e.g., a BiKE, a TriKE, a CAR, etc.). As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the anti-CLEC12A biologic without causing any undesirable biological effects or interacting in a deleterious manner with any of the other components of the pharmaceutical composition in which it is contained.

An anti-CLEC12A biologic may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

Thus, an anti-CLEC12A biologic may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the anti-CLEC12A biologic into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

Thus, in another aspect, this disclosure describes a method of treating any condition where targeting cells that overexpress CLEC12A has therapeutic benefit. In many embodiments, the condition can be a myeloid malignancy. In other embodiments, however, the condition may be an autoimmune condition—e.g., a condition in which myeloid cells infiltrate the central nervous system. Generally, the method includes administering to the subject an amount of the anti-CLEC12A biologic effective for treating the condition. “Treat” or variations thereof refer to reducing, limiting progression, ameliorating, or resolving, to any extent, the symptoms or signs related to a condition. As used herein, “ameliorate” refers to any reduction in the extent, severity, frequency, and/or likelihood of a symptom or clinical sign characteristic of a particular condition; “symptom” refers to any subjective evidence of disease or of a patient's condition; and “sign” or “clinical sign” refers to an objective physical finding relating to a particular condition capable of being found by one other than the patient.

A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is initiated before a condition manifests in a subject. Thus, in certain embodiments, the method can involve prophylactic treatment of a subject at risk of developing a condition. “At risk” refers to a subject that may or may not actually possess the described risk. Thus, for example, a subject “at risk” for developing a specified condition is a subject that possesses one or more indicia of increased risk of having, or developing, the specified condition compared to individuals who lack the one or more indicia, regardless of the whether the subject manifests any symptom or clinical sign of having or developing the condition. Exemplary indicia of a condition can include, for example, genetic predisposition, ancestry, age, sex, geographical location, lifestyle, or medical history. Treatment may also be continued after symptoms have resolved, for example to prevent or delay their recurrence.

In some embodiments, “prophylactic” treatment also includes treatment in the setting of relapse. In other words, a subject may have once manifested symptoms or clinical signs of a condition, but received successful treatment so that the condition is considered to be in remission. Such as subject may no longer manifest a symptom or clinical sign of the condition in remission, but be “at risk” of relapse. In such a context, treatment that includes the anti-CLEC12A biologic may be considered “prophylactic” to reduce the likelihood and/or severity of relapse.

Accordingly, an anti-CLEC12A biologic may be administered to the subject before, during, or after the subject first exhibits a symptom or clinical sign of the condition. Treatment initiated before the subject first exhibits a symptom or clinical sign associated with the condition may result in decreasing the likelihood that the subject experiences clinical evidence of the condition compared to a subject to which the anti-CLEC12A biologic is not administered, decreasing the severity of symptoms and/or clinical signs of the condition, and/or completely resolving the condition. Treatment initiated before the subject first exhibits a symptom or clinical sign associated with relapse of the condition may result in decreasing the likelihood that the subject experiences clinical evidence of relapse compared to a subject to which the anti-CLEC12A biologic is not administered, decreasing the severity of symptoms and/or clinical signs of relapse, and/or completely resolving the condition. Treatment initiated while the subject exhibits a symptom or clinical sign associated with the condition may result in decreasing the severity of symptoms and/or clinical signs of the condition compared to a subject to which the anti-CLEC12A biologic is not administered, and/or completely resolving the condition.

The amount of an anti-CLEC12A biologic administered can vary depending on various factors including, but not limited to, the specific anti-CLEC12A biologic being administered, the weight, physical condition, and/or age of the subject, and/or the route of administration. Thus, the absolute weight of anti-CLEC12A biologic included in a given unit dosage form can vary widely, and depends upon factors such as the species, age, weight and physical condition of the subject, and/or the method of administration. Accordingly, it is not practical to set forth generally the amount that constitutes an amount of anti-CLEC12A biologic effective for all possible applications. Those of ordinary skill in the art, however, can readily determine the appropriate amount with due consideration of such factors.

In some embodiments, the method can include administering sufficient anti-CLEC12A biologic to provide a dose of, for example, from about 100 ng/kg/day to about 50 mg/kg/day to the subject, although in some embodiments the methods may be performed by administering anti-CLEC12A biologic in a dose outside this range.

In some embodiments, the method can includes administering sufficient anti-CLEC12A biologic to provide a minimum dose of at least 100 ng/kg/day such as, for example, at least 1 μg/kg/day, at least 5 μg/kg/day, at least 10 μg/kg/day, at least 25 μg/kg/day, at least 50 μg/kg/day, at least 100 μg/kg/day, at least 200 μg/kg/day, at least 300 μg/kg/day, at least 400 μg/kg/day, at least 500 μg/kg/day, at least 600 μg/kg/day, at least 700 μg/kg/day, at least 800 μg/kg/day, at least 900 μg/kg/day, or at least 1 mg/kg/day.

In some embodiments, the method includes administering sufficient anti-CLEC12A biologic to provide a maximum dose of no more than 10 mg/kg/day such as, for example, no more than 5 mg/kg/day, no more than 4 mg/kg/day, no more than 3 mg/kg/day, no more than 2 mg/kg/day, no more than 1 mg/kg/day, no more than 900 μg/kg/day, no more than 800 μg/kg/day, no more than 700 μg/kg/day, no more than 600 μg/kg/day, no more than 500 μg/kg/day, no more than 400 μg/kg/day, no more than 300 μg/kg/day, no more than 200 μg/kg/day, no more than 100 μg/kg/day, no more than 90 μg/kg/day, no more than 80 μg/kg/day, no more than 70 μg/kg/day, no more than 60 μg/kg/day, no more than 50 μg/kg/day, no more than 40 μg/kg/day, no more than 30 μg/kg/day, no more than 20 μg/kg/day, or no more than 10 μg/kg/day. The anti-CLEC12A biologic provides a dose of “no greater than” a specified amount when the anti-CLEC12A biologic is not absent but is present in an amount up to and including the specified amount.

In some embodiments, the method includes administering sufficient anti-CLEC12A biologic to provide a dose characterized by a range having endpoints defined by any a minimum dose identified above and any maximum dose that is greater than the selected minimum dose. For example, in some embodiments, the method can include administering sufficient anti-CLEC12A biologic to provide a dose of from about 10 μg/kg/day to about 10 mg/kg/day to the subject, a dose of from about 100 μg/kg/day to about 1 mg/kg/day, a dose of from 5 μg/kg/day to 100 μg/kg/day, etc.

In certain embodiments, the method includes administering sufficient anti-CLEC12A biologic to provide a dose that is equal to any minimum dose or any maximum dose listed above. Thus, for example, in certain embodiments, the method can include administering sufficient anti-CLEC12A biologic to provide a dose of 1 μg/kg/day, 5 μg/kg/day, 10 μg/kg/day, 25 μg/kg/day, 50 μg/kg/day, 100 μg/kg/day, 200 μg/kg/day, 500 μg/kg/day, 1 mg/kg/day, 5 mg/kg/day, etc.

In some embodiments, an anti-CLEC12A biologic may be administered, for example, from a single dose to multiple doses per week, although in some embodiments the method can be performed by administering an anti-CLEC12A biologic at a frequency outside this range. In certain embodiments, an anti-CLEC12A biologic may be administered from about once per month to about five times per week. In some embodiments, the doses indicated above, which are described in terms of the amount of anti-CLEC12A biologic administered over a 24-hour period, are administered in a seven-day cycle of four days of treatment and three days of rest.

In some embodiments, an anti-CLEC12A biologic may be administered, for example, from a single dose to multiple cycles of treatment, although in some embodiments the method can be performed by administering an anti-CLEC12A biologic for a duration outside this range. In some embodiments, the anti-CLEC12A biologic may be administered for three weeks. In such embodiments, each week may be a treatment cycle such as the exemplary treatment cycle described in the preceding paragraph. In other embodiments, the anti-CLEC12A biologic may be administered for a greater number of treatment cycles, without a gap between one set of treatment cycles and a subsequent set of treatment cycles. The gap between one set of treatment cycles and a subsequent set of treatment cycles may be a gap of one or more weeks, one or more months, or one or more years.

In some embodiments, the method further includes administering one or more additional therapeutic agents. The one or more additional therapeutic agents (e.g., chemotherapeutic agents) may be administered before, after, and/or coincident to the administration of an anti-CLEC12A biologic. An anti-CLEC12A biologic and the additional therapeutic agents may be co-administered. As used herein, “co-administered” refers to two or more components of a combination administered so that the therapeutic or prophylactic effects of the combination can be greater than the therapeutic or prophylactic effects of either component administered alone. Two components may be co-administered simultaneously or sequentially. Simultaneously co-administered components may be provided in one or more pharmaceutical compositions. Sequential co-administration of two or more components includes cases in which the components are administered so that each component can be present at the treatment site at the same time. Alternatively, sequential co-administration of two components can include cases in which at least one component has been cleared from a treatment site, but at least one cellular effect of administering the component (e.g., cytokine production, activation of a certain cell population, etc.) persists at the treatment site until one or more additional components are administered to the treatment site. Thus, a co-administered combination can, in certain circumstances, include components that never exist in a chemical mixture with one another. In other embodiments, the anti-CLEC12A biologic and the additional therapeutic agent may be administered as part of a mixture or cocktail. In some aspects, the administration of anti-CLEC12A biologic may allow for the effectiveness of a lower dosage of other therapeutic modalities when compared to the administration of the other therapeutic agent or agents alone, thereby decreasing the likelihood, severity, and/or extent of the toxicity observed when a higher dose of the other therapeutic agent or agents is administered.

Exemplary additional therapeutic agents include altretamine, amsacrine, L-asparaginase, colaspase, bleomycin, busulfan, capecitabine, carboplatin, carmustine, chlorambucil, cisplatin, cladribine, cyclophosphamide, cytophosphane, cytarabine, dacarbazine, dactinomycin, daunorubicin, docetaxel, doxorubicin, epirubicin, etoposide, fluorouracil, fludarabine, fotemustine, ganciclovir, gemcitabine, hydroxyurea, idarubicin, ifosfamaide, irinotecan, lomustine, melphalan, mercaptopurine, methotrexate, mitoxantrone, mitomycin C, nimustine, oxaliplatin, paclitaxel, pemetrexed, procarbazine, raltitrexed, temozolomide, teniposide, tioguanine, thiotepa, topotecan, vinblastine, vincristine, vindesine, and vinorelbine.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

### Examples

### Construction of the CD16-huCAMCLEC12A Bi-Specific Compound (SEQ ID NO:34)

Constructs encoding the CD16-huCAMCLEC12A bi-specific compounds were synthesized using PCR and HiFi cloning techniques as previously described (Vallera et al., 2016, Clin Cancer Res 22:3440-3450). Each fully assembled fragment included an EcoRI restriction site, an ATG start codon, the coding sequence for a humanized camelid anti-CD16 sdAb (Vincke et al., 2007, Protein Eng Des Sel 21:1-10), the coding sequence for a huCAMCLEC12A sdAb (one of SEQ ID NOs:16-28), and a sequence encoding a 10×His tag. The assembled fragments were cloned into the Minicircle DNA vector (System Biosciences, LLC, Palo Alto, CA) under the control of a CMV promoter. The DNA sequence was validated to confirm sequence and location of gene insertion (Biomedical Genomics Center, University of Minnesota, Minneapolis, MN).

### Construction of the CD16-huCAMCLEC12A Tri-Specific Compound (SEQ ID NO:35)

Constructs encoding the CD16-huCAMCLEC12A tri-specific compounds were synthesized using PCR and HiFi cloning techniques as previously described (Vallera et al., 2016, Clin Cancer Res 22:3440-3450). Each fully assembled fragment included an EcoRI restriction site, an ATG start codon, the coding sequence for a humanized camelid anti-CD16 sdAb (Vincke et al., 2007, Protein Eng Des Sel 21:1-10), a linker, the coding sequence for a human IL-15 fragment (amino acids 162-175 of SEQ ID NO:35), the coding sequence for a whitlow amino acid linker (GSTSGSGKPGSGEGSTKG; SEQ ID NO:36), the coding sequence for a huCAMCLEC12A sdAb (one of SEQ ID NOs:16-28), and a sequence encoding a 10×His tag. The assembled fragments were cloned into the Minicircle DNA vector (System Biosciences, LLC, Palo Alto, CA) under the control of a CMV promoter. The DNA sequence was validated to confirm sequence and location of gene insertion (Biomedical Genomics Center, University of Minnesota, Minneapolis, MN).

Production and Isolation of CD16-huCAMCLEC12A Bi-Specific Compounds and CD16-huCAMCLEC12A Tri-Specific Compound

Plasmids for all clones were transfected into Expi293 cells (Thermo Fisher Scientific, Inc., Waltham, MA) according to the manufacturer's protocol. The supernatants were collected, and protein was purified using HISPUR cobalt resin (Thermo Fisher Scientific, Inc., Waltham, MA) and PIERCE centrifuge columns (Thermo Fisher Scientific, Inc., Waltham, MA). The protein was eluted using 250 mM imidazole solution and desalted using prepacked disposable PD-10 columns (GE Healthcare Systems, Chicago, IL). Purity and size were determined by running sodium dodecyl sulfate polyacrylamide gel electrophoresis using SIMPLY BLUE LIFE STAIN (Invitrogen, Carlsbad, CA).

### Cancer Cells Lines (HL60 and Raji)

HL60 promyeloblast cells (ATCC CCL-240, American Type Culture Collection, Manassas, VA) where obtained from ATCC and used as a CLEC12A-expressing line. Raji Burkitt's lymphoma lymphoblasts (ATCC CCL-86, American Type Culture Collection, Manassas, VA) were also obtained from ATCC and used as a CLEC12A-negative line.

### Functional Evaluation of Different Clones Using PBMCs Against CLEC12A-Positive HL60 Cells

Healthy donor blood was obtained from Memorial Blood Bank (Minneapolis, MN) and processed to obtain peripheral blood mononuclear cells (PBMCs) using density gradient Ficoll-Paque (GE Healthcare Systems, Chicago, IL). Assessment of NK cell function by flow cytometry was carried out as previously described (Vallera et al., 2016, Clin Cancer Res 22:3440-3450). PBMCs, HL60 cells, and treatments (30 nM) were co-cultured and stained with FITC conjugated anti-CD107a (H4A3, BioLegend, San Diego, CA). One hour after the addition of anti-CD107a, cells were given Golgi Stop and Golgi Plug (BD Biosciences, San Jose, CA) and incubated for three hours. At the end of the incubation, cells were stained with Live/Dead Fixable Aqua Staining Kit (Thermo Fisher Scientific, Inc., Waltham, MA), PE-CY7-conjugated anti-CD56, PE-CF594-conjugated anti-CD3, and PE-conjugated anti-CD69 (FN50, BioLegend, San Diego, CA), fixed, and permeabilized. Permeabilized cells were stained with BV650 conjugated IFNγ (4S.B3, BioLegend, San Diego, CA) and expression was evaluated by flow cytometry.

### Evaluation of Functional Specificity of Clone 33 Using NK Cells Against Nothing, CLEC12A-Positive HL60 Cells, or CLEC12A-Negative Raji Cells

Healthy donor blood was obtained from Memorial Blood Bank (Minneapolis, MN) and processed to obtain peripheral blood mononuclear cells (PBMCs) using density gradient FICOLL-PAQUE (GE Healthcare Systems, Chicago, IL). PBMCs were used to magnetically enrich NK cells using an EASYSEP human NK cell enrichment kit (STEMCELL Technologies, Inc., Vancouver, BC). Assessment of NK cell function by flow cytometry was carried out as previously described (Vallera et al., 2016, Clin Cancer Res 22:3440-3450). Enriched NK cells were incubated alone, with HL60 cells, or with Raji cells and noted treatments (30 nM). Cells and treatments were co-cultured and stained with FITC conjugated anti-CD107a (H4A3, BioLegend, San Diego, CA). One hour after the addition of anti-CD107a, cells were given Golgi Stop and Golgi Plug (BD Biosciences, San Jose, CA) and incubated for three hours. At the end of the incubation, cells were stained with Live/Dead Fixable Aqua Staining Kit (Thermo Fisher Scientific, Inc., Waltham, MA), PE-CY7-conjugated anti-CD56, PE-CF594-conjugated anti-CD3, and PE-conjugated anti-CD69 (BioLegend, San Diego, CA), fixed, and permeabilized. Permeabilized cells were stained with BV650-conjugated IFNγ (BioLegend, San Diego, CA) and expression was evaluated by flow cytometry.

### Determination of Binding Specificity

To determine binding specificity, 30 nM of the Clone 33 bi-specific compound or the anti-CLEC12A scFv-containing tri-specific compound were incubated with CLEC12A-positive HL60 cells or CLEC12A-negative Raji cells for 30 minutes at 37° C. The cells were spun down and washed twice. An anti-HIS, phycoerythrin (PE)-labeled, antibody (OriGene Technologies, Inc., Rockville, MD) was added and incubated with cells for 20 minutes at 4° C. Cells were washed twice, fixed with 2% paraformaldehyde, and run on a flow cytometer to evaluate percent of cells binding to noted constructs.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text

SEQ ID NO: 1-CLEC12A clone 1

| | |
|---|---|
| 1 | QVQLQESGGG LVQPGGSLRL SCAASGDRES NDVMAWVRQA PGKGLEWVSA |
| 51 | IVTQDGSTYY ADSVKGRFTI SRDNSKNTLY LQMNSLRAED TAVYYCAGEP |
| 101 | LDAEDLWYWG QGTLVTVSS |

SEQ ID NO: 2-CLEC12A clone 3

| | |
|---|---|
| 1 | QVQLLESGGG LVQPGGSLRL SCAASGVRVS AQFMSWVRQA PGKGLEWVSS |
| 51 | IYKKNGSTYY ADSVKGRFTI SRDNSKNTLY LQMNSLRAED TAVYYCAIDH |
| 101 | GEEHKELYSW GQGTLVTVSS |

SEQ ID NO: 3-CLEC12A clone 9

| | |
|---|---|
| 1 | QVQLLESGGG LVQPGGSLRL SCAASGYSIT DQDMSWVRQA PGKGLEWVSG |
| 51 | ILATSGSTYY ADSVKGRFTI SRDNSKNTLY LQMNSLRAED TAVYYCAGEV |
| 101 | EKSSQSMPFW GQGTLVTVSS |

SEQ ID NO: 4-CLEC12A clone 10

| | |
|---|---|
| 1 | QVQLLESGGG LVQPGGSLRL SCAASGVNVT ADDMSWVRQA PGKGLEWVST |
| 51 | IDGGDGSTYY ADSVKGRFTI SRDNSKNTLY LQMNSLRAED TAVYYCATAF |
| 101 | TSHEAEVHSW GQGTLVTVSS |

SEQ ID NO: 5-CLEC12A clone 11

| | |
|---|---|
| 1 | QVQLLESGGG LVQPGGSLRL SCAASGYRLS NDIMAWVRQA PGKGLEWVSA |
| 51 | IVDTDGSTYY ADSVKGRFTI SRDNSKNTLY LQMNSLRAED TAVYYCARDP |
| 101 | YNEDDVSYWG QGTLVTVSS |

SEQ ID NO: 6-CLEC12A clone 17

| | |
|---|---|
| 1 | QVQLLESGGG LVQPGGSLRL SCAASGERFI AQDMSWVRQA TGKGLEWVST |
| 51 | IATNDGSTYY ADSVKGRFTI SRDNSKNTLY LQMNSLRAED TAVYYCATDS |
| 101 | SWENDLWYWG QGTLVTVSS |

SEQ ID NO: 7-CLEC12A clone 23

| | |
|---|---|
| 1 | QVQLLESGGG LVQPGGSLRL SCAASGERLT DEDMSWVRQA PGKGLEWVST |
| 51 | IATRDGSTYY ADSVKGRFTI SRDNSKNTLY LQMNSLRAED TAVYYCAGLR |
| 101 | DQLYGNEQLA FWGQGTLVTV SS |

SEQ ID NO: 8-CLEC12A clone 29

| | |
|---|---|
| 1 | QVQLLESGGG LVQPGGSLRL SCAASGYNVI YEDMGWVRQA PGKGLEWVSG |
| 51 | IDVPSGSTYY ADSVKGRFTI SRDNSKNTLY LQMNSLRAED TAVYYCAAVV |
| 101 | GQDWFQAEPS EMYYWGQGTL VTVSS |

SEQ ID NO: 9-CLEC12A clone 33

| | |
|---|---|
| 1 | QVQLLESGGG LVQPGGSLRL SCAASGDMES YDDMGWVRQA PGKGLEWVSG |
| 51 | IQNTDGSTYY ADSVKGRFTI SRDNSKNTLY LQMNSLRAED TAVYYCATLY |
| 101 | DRMVGKEEQL ASWGQGTLVT VSS |

SEQ ID NO: 10-CLEC12A clone 40

| | |
|---|---|
| 1 | QVQLLESGGG LVQPGGSLRL SCAASGDTIN PEDMGWVRQA PGKGLEWVSA |
| 51 | IEAQSGSTYY ADSVKGRFTI SRDNSKNTLY LQMNSLRAED TAVYYCARLT |
| 101 | AHQEEPVAYW GQGTLVTVSS |

SEQ ID NO: 11-CLEC12A clone 56

| | |
|---|---|
| 1 | QVQLLESGGG LVQPGGSLRL SCAASGFTVT DHDMGWVRQA PGKGLEWVSS |
| 51 | ITVGNGSTYY ADSVKGRFTI SRDNSKNTLY LQMNSLRAED TAVYYCATEP |
| 101 | LPHQEVTYWG QGTLVTVSS |

SEQ ID NO: 12-CLEC12A clone 60

| | |
|---|---|
| 1 | QVQLLESGGG LVQPGGSLRL SCAASGYMFS ADVMSWVRQA PGKGLEWVSG |
| 51 | IWIPDGSTYY ADSVKGRFTI SRDNSKNTLY LQMNSLRAED TAVYYCAAQL |
| 101 | NERIASITKN MHSWGQGTLV TVSS |

SEQ ID NO: 13-CLEC12A clone 86

| | |
|---|---|
| 1 | QVQLLESGGG LVQPGGSLRL SCAASGDMLS AQDMGWVRQA PGKGLEWVSG |
| 51 | IDSDDGSTYY ADSVKGRETI SRDNSKNTLY LQMNSLRAED TAVYYCAALW |
| 101 | GEDVIMQDSS VGSWGQGTLV TVSS |

SEQ ID NO: 14-CLEC12A clone consensus sequence

| | |
|---|---|
| 1 | QVQLLESGGG LVQPGGSERL SCAASGYXVS XDDMSWVRQA PGKGLEWVSA |
| 51 | IXXXDGSTYY ADSVKGRFTI SRDNSKNTLY LQMNSLRAED TAVYYCAXXX |
| 101 | XXXXXXXXXX LXYWGQGTLV TVSS |

X = any amino acid or no amino acid
SEQ ID NO: 15-CLEC12A TriKE amino acid sequence

| | |
|---|---|
| 1 | MKWVTFISLL FLESSAYSQV QLVESGGGLV QPGGSLRLSC AASGLTESSY |
| 51 | NMGWFRQAPG QGLEAVASIT WSGRDTFYAD SVKGRFTISR DNSKNTLYLQ |
| 101 | MNSLRAEDTA VYYCAANPWP VAAPRSGTYW GQGTLVTVSS SGGGGSGGGG |
| 151 | SGGGGSGGGG SGNWVNVISD LKKIEDLIQS MHIDATLYTE SDVHPSCKVT |
| 201 | AMKCFLLELQ VISLESGDAS IHDTVENLII LANNSLSSNG NVTESGCKEC |
| 251 | EELEEKNIKE FLQSFVHIVQ MFINTSGSTS GSGKPGSGEG STKGQVQLQE |
| 301 | SGPGLVKPSE TLSLTCVVSG GSISSSNWWS WVRQPPGKGL EWIGEIYHSG |
| 351 | SPDYNPSLKS RVTISVDKSR NQFSLKLSSV TAADTAVYYC AKVSTGGFFD |
| 401 | YWGQGTLVTV SSGGGGSGGG GSGGGGSEIE LTQSPSSLSA SVGDRVTITC |
| 451 | RASQSISSYL NWYQQKPGKA PKLLIYAASS LQSGVPSRFS GSGSGTDFTL |
| 501 | TISSLQPEDF ATYYCQQSYS TPPTFGPGTK VEI |

underlined: signal peptide
bold: anti-CLEC12A scFv
SEQ ID NO: 16-Clone 1 DNA sequence

| | |
|---|---|
| 1 | CAGGTGCAGC TGCAGGAGTC TGGGGGAGGC TTGGTACAGC CTGGGGGGTC CCTGCGTCTC |
| 61 | TCCTGTGCAG CCTCCGGAGA TAGGTTTAGC AATGACGTTA TGGCCTGGGT CCGCCAGGCT |
| 121 | CCAGGGAAGG GTCTAGAGTG GGTATCAGCC ATTGTTACCC AAGACGGTAG CACATACTAC |
| 181 | GCAGACTCCG TGAAGGGCCG GTTCACCATC TCCCGTGACA ATTCCAAGAA CACGCTGTAT |
| 241 | CTGCAAATGA ACAGCCTGCG TGCCGAGGAC ACCGCGGTAT ATTATTGCGC GGGAGAGCCT |
| 301 | CTTGATGCCG AGGACTTGTG GTATTGGGGT CAGGGAACCC TGGTCACCGT CTCGAGC |

SEQ ID NO: 17-Clone 3 DNA sequence

| | |
|---|---|
| 1 | CAGGTGCAGC TGTTGGAGTC TGGGGGAGGC TTGGTACAGC CTGGGGGGTC CCTGCGTCTC |
| 61 | TCCTGTGCAG CCTCCGGAGT TAGGGTTAGC GCTCAGTTTA TGAGCTGGGT CCGCCAGGCT |
| 121 | CCAGGGAAGG GTCTAGAGTG GGTATCAAGC ATTTATAAGA AAAACGGTAG CACATACTAC |
| 181 | GCAGACTCCG TGAAGGGCCG GTTCACCATC TCCCGTGACA ATTCCAAGAA CACGCTGTAT |
| 241 | CTGCAAATGA ACAGCCTGCG TGCCGAGGAC ACCGCGGTAT ATTATTGCGC GATTGATCAT |
| 301 | GGGGAGGAGC ACAAGGAGCT GTACTCTTGG GGTCAGGGAA CCCTGGTCAC CGTCTCGAGC |

SEQ ID NO: 18-Clone 9 DNA sequence

| | |
|---|---|
| 1 | CAGGTGCAGC TGTTGGAGTC TGGGGGAGGC TTGGTACAGC CTGGGGGGTC CCTGCGTCTC |
| 61 | TCCTGTGCAG CCTCCGGATA TAGCATTACC GATCAGGATA TGAGCTGGGT CCGCCAGGCT |
| 121 | CCAGGGAAGG GTCTAGAGTG GGTATCAGGC ATTCTGGCCA CAAGCGGTAG CACATACTAC |
| 181 | GCAGACTCCG TGAAGGGCCG GTTCACCATC TCCCGTGACA ATTCCAAGAA CACGCTGTAT |
| 241 | CTGCAAATGA ACAGCCTGCG TGCCGAGGAC ACCGCGGTAT ATTATTGCGC GGGAGAGGTT |
| 301 | GAGAAGAGTT CCCAGTCGAT GCCGTTTTGG GGTCAGGGAA CCCTGGTCAC CGTCTCGAGC |

SEQ ID NO: 19-Clone 10 DNA sequence

| | |
|---|---|
| 1 | CAGGTGCAGC TGTTGGAGTC TGGGGGAGGC TTGGTACAGC CTGGGGGGTC CCTGCGTCTC |
| 61 | TCCTGTGCAG CCTCCGGAGT TAACGTTACC GCTGACGATA TGAGCTGGGT CCGCCAGGCT |
| 121 | CCAGGGAAGG GTCTAGAGTG GGTATCAACC ATTGATGGCG GTGACGGTAG CACATACTAC |
| 181 | GCAGACTCCG TGAAGGGCCG GTTCACCATC TCCCGTGACA ATTCCAAGAA CACGCTGTAT |
| 241 | CTGCAAATGA ACAGCCTGCG TGCCGAGGAC ACCGCGGTAT ATTATTGCGC GACTGCTTTT |
| 301 | ACGTCGCATG AGGCGGAGGT CCACTCTTGG GGTCAGGGAA CCCTGGTCAC CGTCTCGAGC |

SEQ ID NO: 20-Clone 11 DNA sequence

| | |
|---|---|
| 1 | CAGGTGCAGC TGTTGGAGTC TGGGGGAGGC TTGGTACAGC CTGGGGGGTC CCTGCGTCTC |
| 61 | TCCTGTGCAG CCTCCGGATA TAGGCTTAGC AATGACATTA TGGCCTGGGT CCGCCAGGCT |
| 121 | CCAGGGAAGG GTCTAGAGTG GGTATCAGCC ATTGTGGACA CAGACGGTAG CACATACTAC |
| 181 | GCAGACTCCG TGAAGGGCCG GTTCACCATC TCCCGTGACA ATTCCAAGAA CACGCTGTAT |
| 241 | CTGCAAATGA ACAGCCTGCG TGCCGAGGAC ACCGCGGTAT ATTATTGCGC GAGAGATCCT |
| 301 | TATAATGAGG ACGACGTCAG CTATTGGGGT CAGGGAACCC TGGTCACCGT CTCGAGC |

SEQ ID NO: 21-Clone 17 DNA sequence

| | |
|---|---|
| 1 | CAGGTGCAGC TGTTGGAGTC TGGGGGAGGC TTGGTACAGC CTGGGGGGTC CCTGCGTCTC |
| 61 | TCCTGTGCAG CCTCCGGATT TAGGTTTATC GCTCAGGATA TGAGCTGGGT CCGCCAGGCT |
| 121 | ACAGGGAAGG GTCTAGAGTG GGTATCAACC ATTGCTACGA ATGACGGTAG CACATACTAC |
| 181 | GCAGACTCCG TGAAGGGCCG GTTCACCATC TCCCGTGACA ATTCCAAGAA CACGCTGTAT |
| 241 | CTGCAAATGA ACAGCCTGCG TGCCGAGGAC ACCGCGGTAT ATTATTGCGC GACTGATTCG |
| 301 | TCTTGGGAGA ACGACTTGTG GTATTGGGGT CAGGGAACCC TGGTCACCGT CTCGAGC |

SEQ ID NO: 22-Clone 23 DNA sequence

| | |
|---|---|
| 1 | CAGGTGCAGC TGTTGGAGTC TGGGGGAGGC TTGGTACAGC CTGGGGGGTC CCTGCGTCTC |
| 61 | TCCTGTGCAG CCTCCGGATT TAGGCTTACC GATGAGGATA TGAGCTGGGT CCGCCAGGCT |
| 121 | CCAGGGAAGG GTCTAGAGTG GGTATCAACC ATTGCTACCA GAGACGGTAG CACATACTAC |
| 181 | GCAGACTCCG TGAAGGGCCG GTTCACCATC TCCCGTGACA ATTCCAAGAA CACGCTGTAT |
| 241 | CTGCAAATGA ACAGCCTGCG TGCCGAGGAC ACCGCGGTAT ATTATTGCGC GGGACTTAGG |
| 301 | GATCAGTTGT ATGGGAACGA GCAGTTGGCC TTTTGGGGTC AGGGAACCCT GGTCACCGTC |
| 361 | TCGAGC |

SEQ ID NO: 23-Clone 29 DNA sequence

| | |
|---|---|
| 1 | CAGGTGCAGC TGTTGGAGTC TGGGGGAGGC TTGGTACAGC CTGGGGGGTC CCTGCGTCTC |
| 61 | TCCTGTGCAG CCTCCGGATA TAACGTTATC TATGAGGATA TGGGCTGGGT CCGCCAGGCT |
| 121 | CCAGGGAAGG GTCTAGAGTG GGTATCAGGC ATTGATGTCC CTAGCGGTAG CACATACTAC |
| 181 | GCAGACTCCG TGAAGGGCCG GTTCACCATC TCCCGTGACA ATTCCAAGAA CACGCTGTAT |
| 241 | CTGCAAATGA ACAGCCTGCG TGCCGAGGAC ACCGCGGTAT ATTATTGCGC GGCTGTTGTT |
| 301 | GGTCAGGATT GGTTTCAGGC TGAGCCCTCG GAGATGTACT ATTGGGGTCA GGGAACCCTG |
| 361 | GTCACCGTCT CGAGC |

SEQ ID NO: 24-Clone 33 DNA sequence

| | |
|---|---|
| 1 | CAGGTGCAGC TGTTGGAGTC TGGGGGAGGC TTGGTACAGC CTGGGGGGTC CCTGCGTCTC |
| 61 | TCCTGTGCAG CCTCCGGAGA TATGTTTAGC TATGACGATA TGGGCTGGGT CCGCCAGGCT |
| 121 | CCAGGGAAGG GTCTAGAGTG GGTATCAGGC ATTCAGAACA CTGACGGTAG CACATACTAC |
| 181 | GCAGACTCCG TGAAGGGCCG GTTCACCATC TCCCGTGACA ATTCCAAGAA CACGCTGTAT |
| 241 | CTGCAAATGA ACAGCCTGCG TGCCGAGGAC ACCGCGGTAT ATTATTGCGC GACTCTGTAT |
| 301 | GATAGGATGG TTGGGAAGGA GGAGCAGTTG GCCTCTTGGG GTCAGGGAAC CCTGGTCACC |
| 361 | GTCTCGAGC |

SEQ ID NO: 25-Clone 40 DNA sequence

| | |
|---|---|
| 1 | CAGGTGCAGC TGTTGGAGTC TGGGGGAGGC TTGGTACAGC CTGGGGGGTC CCTGCGTCTC |
| 61 | TCCTGTGCAG CCTCCGGAGA TACCATTAAC CCTGAGGATA TGGGCTGGGT CCGCCAGGCT |
| 121 | CCAGGGAAGG GTCTAGAGTG GGTATCAGCC ATTGAGGCGC AAAGCGGTAG CACATACTAC |
| 181 | GCAGACTCCG TGAAGGGCCG GTTCACCATC TCCCGTGACA ATTCCAAGAA CACGCTGTAT |
| 241 | CTGCAAATGA ACAGCCTGCG TGCCGAGGAC ACCGCGGTAT ATTATTGCGC GAGATTGACT |
| 301 | GCTCATCAGG AGGAGCCGGT CGCGTATTGG GGTCAGGGAA CCCTGGTCAC CGTCTCGAGC |

SEQ ID NO: 26-Clone 56 DNA sequence

| | |
|---|---|
| 1 | CAGGTGCAGC TGTTGGAGTC TGGGGGAGGC TTGGTACAGC CTGGGGGGTC CCTGCGTCTC |
| 61 | TCCTGTGCAG CCTCCGGATT TACGGTTACC GATCACGATA TGGGCTGGGT CCGCCAGGCT |
| 121 | CCAGGGAAGG GTCTAGAGTG GGTATCAAGC ATTACTGTGG GAAACGGTAG CACATACTAC |
| 181 | GCAGACTCCG TGAAGGGCCG GTTCACCATC TCCCGTGACA ATTCCAAGAA CACGCTGTAT |
| 241 | CTGCAAATGA ACAGCCTGCG TGCCGAGGAC ACCGCGGTAT ATTATTGCGC GACAGAGCCT |
| 301 | TTGCCGCACC AGGAGGTCAC GTATTGGGGT CAGGGAACCC TGGTCACCGT CTCGAGC |

SEQ ID NO: 27-Clone 60 DNA sequence

| | |
|---|---|
| 1 | CAGGTGCAGC TGTTGGAGTC TGGGGGAGGC TTGGTACAGC CTGGGGGGTC CCTGCGTCTC |
| 61 | TCCTGTGCAG CCTCCGGATA TATGTTTAGC GCTGACGTTA TGAGCTGGGT CCGCCAGGCT |
| 121 | CCAGGGAAGG GTCTAGAGTG GGTATCAGGC ATTTGGATCC CTGACGGTAG CACATACTAC |
| 181 | GCAGACTCCG TGAAGGGCCG GTTCACCATC TCCCGTGACA ATTCCAAGAA CACGCTGTAT |
| 241 | CTGCAAATGA ACAGCCTGCG TGCCGAGGAC ACCGCGGTAT ATTATTGCGC GGCTCAGCTG |
| 301 | AATGAGAGGA TTGCTTCTAT TACGAAGAAC ATGCACTCTT GGGGTCAGGG AACCCTGGTC |
| 361 | ACCGTCTCGA GC |

SEQ ID NO: 28-Clone 86 DNA sequence

| | |
|---|---|
| 1 | CAGGTGCAGC TGTTGGAGTC TGGGGGAGGC TTGGTACAGC CTGGGGGGTC CCTGCGTCTC TCCTGTGCAG CCTCCGGAGA TATGCTTAGC GCTCAGGATA TGGGCTGGGT CCGCCAGGCT |
| 61 | CCAGGGAAGG GTCTAGAGTG GGTATCAGGC ATTGATAGCG ATGACGGTAG CACATACTAC |
| 121 | GCAGACTCCG TGAAGGGCCG GTTCACCATC TCCCGTGACA ATTCCAAGAA CACGCTGTAT |
| 181 | CTGCAAATGA ACAGCCTGCG TGCCGAGGAC ACCGCGGTAT ATTATTGCGC GGCATTGTGG |
| 241 | GGGGAGGATG TGATTATGCA GGACTCGTCG GTGGGGTCTT GGGGTCAGGG AACCCTGGTC |
| 301 | ACCGTCTCGA GC |

SEQ ID NO: 29-CDR1
X₁X₂X₃MX₄
X₁ = N, A, D, Y, or P
X₂ = D, E, H, Q, or deletion
X₃ = V, F, I, or D
X₄ = A, S, or G
SEQ ID NO: 30-CDR2
X₁IX₂X₃X₄X₅GSTY YADSVKGRFT
X₁ = A, S, T, or G
X₂ = V, Y, L, D, A, E, T, W, Q, or deletion
X₃ = T, K, A, G, D, V, N, I, or S
X₄ = Q, K, T, G, N, R, P, or D
X₅ = D, N, or S
SEQ ID NO: 31-CDR3
X₁X₂X₃X₄X₅X₆X₇X₈X₉X₁₀X₁₁X₁₂X₁₃X₁₄X₁₅X₁₆
X₁ = any amino acid or deletion
X₂ = any amino acid or deletion
X₃ = any amino acid or deletion
X₄ = P, E, S, Q, R, or H
X₅ = any amino acid or deletion
X₆ = any amino acid or deletion
X₇ = any amino acid or deletion
X₈ = any amino acid or deletion
X₉ = any amino acid or deletion
X₁₀ = any amino acid or deletion
X₁₁ = any amino acid or deletion
X₁₂ = any amino acid or deletion
X₁₃ = any amino acid or deletion
X₁₄ = L, M, or V
X₁₅ = W, Y, P, H, S, A, T, or G
X₁₆ = Y, S, or F
SEQ ID NO: 32-Signal peptide
MKWVTFISLL FLESSAYS
SEQ ID NO: 33-Histidine tag, including spacer
VDEHHHHHHH HHH
SEQ ID NO: 34-CD16-huCAM Clone 33 bi-specific compound
KWVTFISLLF LESSAYSQVQ LVESGGGLVQ PGGSLRLSCA ASGLTESSYN MGWERQAPGQ
GLEAVASITW SGRDTFYADS VKGRFTISRD NSKNTLYLQM NSLRAEDTAV YYCAANPWPV
AAPRSGTYWG QGTLVTVSSS GGGGSGGGGS GGGGSGGGGS GGSQVQLLES GGGLVQPGGS
LRLSCAASGD MFSYDDMGWV RQAPGKGLEW VSGIQNTDGS TYYADSVKGR FTISRDNSKN
TLYLQMNSLR AEDTAVYYCA TLYDRMVGKE EQLASWGQGT LVTVSSGSHH HHHHHHHH
underline: signal sequence
double underline: HuEF91
Bold: anti-CLEC12A Clone 33
SEQ ID NO: 35-CD16-huCAM Clone 33 tri-specific compound
KWVTFISLLF LESSAYSQVQ LVESGGGLVQ PGGSLRLSCA ASGLTESSYN MGWERQAPGO
GLEAVASITW SGRDTFYADS VKGRFTISRD NSKNTLYLQM NSLRAEDTAV YYCAANPWPV
AAPRSGTYWG QGTLVTVSSS GGGGSGGGGS GGGGSGGGGS

[symbol]

[symbol]

[symbol]

[symbol]

[symbol]

[symbol]

GSTSG SGKPGSGEGS TKGGSQVQLL
ESGGGLVQPG GSLRLSCAAS GDMFSYDDMG WVRQAPGKGL EWVSGIQNTD GSTYYADSVK
GRFTISRDNS KNTLYLQMNS LRAEDTAVYY CATLYDRMVG KEEQLASWGQ GTLVTVSSGS
HHHHHHHHHH
underline: signal sequence
double underline: HuEF91
hashed underline: human IL-15 fragment
bold: anti-CLEC12A Clone 33
SEQ ID NO: 36-Whitlow linker
GSTSGSGKPG SGEGSTKG

1. A polypeptide comprising at least 90% amino acid similarity to SEQ ID NO:14.

2. A polypeptide comprising at least 90% amino acid identity to SEQ ID NO:14.

3. The polypeptide of claim 1 comprising:
the amino acid sequence of SEQ ID NO:1;
the amino acid sequence of SEQ ID NO:2;
the amino acid sequence of SEQ ID NO:3;
the amino acid sequence of SEQ ID NO:4;
the amino acid sequence of SEQ ID NO:5;
the amino acid sequence of SEQ ID NO:6;
the amino acid sequence of SEQ ID NO:7;
the amino acid sequence of SEQ ID NO:8;
the amino acid sequence of SEQ ID NO:9;
the amino acid sequence of SEQ ID NO:10;
the amino acid sequence of SEQ ID NO:11;
the amino acid sequence of SEQ ID NO:12; or
the amino acid sequence of SEQ ID NO:13.

4. The polypeptide of claim 1 comprising:
the amino acid sequence of SEQ ID NO:29;
the amino acid sequence of SEQ ID NO:30; and
the amino acid sequence of SEQ ID NO:31.

5. A polypeptide comprising:
the amino acid sequence of SEQ ID NO:29;
the amino acid sequence of SEQ ID NO:30; and
the amino acid sequence of SEQ ID NO:31.

6. A biologic therapeutic compound comprising the polypeptide of claim 1.

7. The biologic therapeutic compound of claim 6, wherein the biologic therapeutic compound is:
a bi-specific Killer engager molecule (BiKE);
a tri-specific Killer engager molecule (TriKE);
a tetra-specific Killer engager molecule (TetraKE);
a penta-specific Killer engager molecule (PentaKE);
a bi-specific T cell engager molecule (BiTE);
a tri-specific T cell engager molecule (TriTE);
tetra-specific T cell engager molecule (TetraTE);
a penta-specific T cell engager molecule (PentaTE);
a chimeric antigen receptor;
a full antibody;
an antibody-drug conjugate (ADC) molecule;
a targeted delivery construct; or
a labeling construct.

8. A pharmaceutical composition comprising:
the biologic therapeutic compound of claim 6; and
a pharmaceutically acceptable carrier.

9. A method comprising:
administering to a subject having a tumor characterized in expression of CLEC12A a biologic therapeutic of claim 6 in an amount to ameliorate at least one symptom or clinical sign of the tumor.

10. The method of claim 9, wherein the biological therapeutic is:
a bi-specific Killer engager molecule (BiKE);
a tri-specific Killer engager molecule (TriKE);
a tetra-specific Killer engager molecule (TetraKE);
a penta-specific Killer engager molecule (PentaKE);
a bi-specific T cell engager molecule (BiTE);
a tri-specific T cell engager molecule (TriTE);
tetra-specific T cell engager molecule (TetraTE);
a penta-specific T cell engager molecule (PentaTE);
a chimeric antigen receptor;
a full antibody;
an antibody-drug conjugate (ADC) molecule; or
a targeted delivery construct.

11. The polypeptide of claim 2 comprising:
the amino acid sequence of SEQ ID NO:1;
the amino acid sequence of SEQ ID NO:2;
the amino acid sequence of SEQ ID NO:3;
the amino acid sequence of SEQ ID NO:4;
the amino acid sequence of SEQ ID NO:5;
the amino acid sequence of SEQ ID NO:6;
the amino acid sequence of SEQ ID NO:7;
the amino acid sequence of SEQ ID NO:8;
the amino acid sequence of SEQ ID NO:9;
the amino acid sequence of SEQ ID NO:10;
the amino acid sequence of SEQ ID NO:11;
the amino acid sequence of SEQ ID NO:12; or
the amino acid sequence of SEQ ID NO:13.

12. The polypeptide of claim 2 comprising:
the amino acid sequence of SEQ ID NO:29;
the amino acid sequence of SEQ ID NO:30; and
the amino acid sequence of SEQ ID NO:31.

13. A biologic therapeutic compound comprising the polypeptide of claim 2.

14. The biologic therapeutic compound of claim 13, wherein the biologic therapeutic compound is:
a bi-specific Killer engager molecule (BiKE);
a tri-specific Killer engager molecule (TriKE);
a tetra-specific Killer engager molecule (TetraKE);
a penta-specific Killer engager molecule (PentaKE);
a bi-specific T cell engager molecule (BiTE);
a tri-specific T cell engager molecule (TriTE);
tetra-specific T cell engager molecule (TetraTE);
a penta-specific T cell engager molecule (PentaTE);
a chimeric antigen receptor;
a full antibody;
an antibody-drug conjugate (ADC) molecule;
a targeted delivery construct; or
a labeling construct.

15. A biologic therapeutic compound comprising the polypeptide of claim 3.

16. The biologic therapeutic compound of claim 15, wherein the biologic therapeutic compound is:
a bi-specific Killer engager molecule (BiKE);
a tri-specific Killer engager molecule (TriKE);
a tetra-specific Killer engager molecule (TetraKE);
a penta-specific Killer engager molecule (PentaKE);
a bi-specific T cell engager molecule (BiTE);
a tri-specific T cell engager molecule (TriTE);
tetra-specific T cell engager molecule (TetraTE);
a penta-specific T cell engager molecule (PentaTE);
a chimeric antigen receptor;
a full antibody;
an antibody-drug conjugate (ADC) molecule;
a targeted delivery construct; or
a labeling construct.

17. A biologic therapeutic compound comprising the polypeptide of claim 4.

18. The biologic therapeutic compound of claim 17, wherein the biologic therapeutic compound is:
a bi-specific Killer engager molecule (BiKE);
a tri-specific Killer engager molecule (TriKE);
a tetra-specific Killer engager molecule (TetraKE);
a penta-specific Killer engager molecule (PentaKE);
a bi-specific T cell engager molecule (BiTE);
a tri-specific T cell engager molecule (TriTE);
tetra-specific T cell engager molecule (TetraTE);
a penta-specific T cell engager molecule (PentaTE);
a chimeric antigen receptor;
a full antibody;
an antibody-drug conjugate (ADC) molecule;
a targeted delivery construct; or
a labeling construct.

19. A biologic therapeutic compound comprising the polypeptide of claim 5.

20. The biologic therapeutic compound of claim 19, wherein the biologic therapeutic compound is:
a bi-specific Killer engager molecule (BiKE);
a tri-specific Killer engager molecule (TriKE);
a tetra-specific Killer engager molecule (TetraKE);
a penta-specific Killer engager molecule (PentaKE);
a bi-specific T cell engager molecule (BiTE);
a tri-specific T cell engager molecule (TriTE);
tetra-specific T cell engager molecule (TetraTE);
a penta-specific T cell engager molecule (PentaTE);
a chimeric antigen receptor;
a full antibody;
an antibody-drug conjugate (ADC) molecule;
a targeted delivery construct; or
a labeling construct.
